(19) United States
(12) Patent Application Publication
(10) Pub. No.: US 2025/0041199 A1
(43) Pub. Date: Feb. 6, 2025

(54) PREPARATION FOR BLEACHING KERATIN FIBERS, CONTAINING AT LEAST ONE AMINOSILICONE AND A WHITE PIGMENT

(71) Applicant: Henkel AG & Co. KGaA, Duesseldorf (DE)

(72) Inventors: Constanze Kruck, Grevenbroich (DE); Gabriele Weser, Essen (DE); Ulrike Schumacher, Düsseldorf (DE); Imme Breuer, Düsseldorf (DE); Melanie Moch, Dormagen (DE); Caroline Kriener, Düsseldorf (DE)

(21) Appl. No.: 18/912,198

(22) Filed: Oct. 10, 2024

Related U.S. Application Data
(63) Continuation of application No. PCT/EP2023/050561, filed on Jan. 11, 2023.

(30) Foreign Application Priority Data
Apr. 12, 2022 (DE) 102022203643.3

(51) Int. Cl. A61K 8/898; A61Q 5/06; A61Q 5/08

(52) U.S. Cl. CPC A61K 8/898; A61Q 5/065; A61Q 5/08; A61K 2800/43; A61K 2800/592; A61K 2800/805

(57) ABSTRACT

A preparation for dyeing keratin fibers, in particular human hair. The preparation includes (a1) at least one amino-functionalized silicone polymer, and (a2) at least one white pigment. The invention also relates to a process for bleaching keratin fibers, wherein the aforementioned preparation is used on the keratin fibers, in particular human hair.

### FIELD OF INVENTION

The subject of the present application is an agent for lightening keratinous fibers—in particular, human hair—which contains at least one amino-functionalized silicone polymer (a1), and at least one white pigment (a2).

A second subject of this application is a method for lightening keratinous fibers—in particular, human hair—wherein an agent of the first subject of the invention is applied to the keratinous fibers, allowed to act, and then optionally washed out again with water.

### BACKGROUND

Changing the shape and color of keratinous fibers—in particular, human hair—represents an important field of modern cosmetics. To change the hair color, the skilled artisan is familiar with a variety of coloring systems depending upon the coloring requirements. Oxidation lightening agents are typically used for permanent, intense coloring with good fastness properties and good gray coverage. Such lightening agents contain oxidation dye precursors, what are known as developer components and coupler components, which together form the actual dyes under the influence of oxidizing agents such as, for example, hydrogen peroxide. Oxidation lightening agents are characterized by very long-lasting color results.

When using direct dyes, dyes which are already formed diffuse out of the lightening agent into the hair fiber. In comparison with oxidative hair coloring, the colors obtained with direct dyes have a lower durability and a more rapid washing out. Colors with direct dyes usually remain on the hair for a period of between 5 and 20 hair washes.

The use of color pigments for brief changes in color on the hair and/or the skin is known. Color pigments are generally understood to mean insoluble dyeing substances. These are present undissolved in the form of small particles in the coloring formulation and are only deposited from the outside onto the hair fibers and/or the skin surface. They can therefore generally be removed again without leaving residue by washing a few times with surfactant-containing cleaning agents. Various products of this type by the name of hair mascara are available on the market.

If the user desires particularly long-lasting coloring, the use of oxidative lightening agents has hitherto been the only option. However, despite multiple optimization attempts, an unpleasant ammonia odor or amine odor cannot be completely avoided in oxidative hair coloring. The hair damage that remains associated with the use of the oxidative lightening agents also has a disadvantageous effect on the hair of the user. The search for alternative, high-performance coloring methods is therefore an ongoing challenge. One possible alternative coloring system, which recently has been moving increasingly into focus, is based upon the use of colored pigments.

Coloring with pigments offers various major advantages. Since the pigments are deposited only from the outside to the keratinous fibers—in particular, to the hair fibers—the damage associated with the dyeing process is very particularly low. Furthermore, desired colorings that are no longer desired can be removed quickly and easily without residue and therefore offer the user the possibility of returning directly and without great effort to their original hair color. This coloring process is therefore particularly attractive for consumers who do not regularly want to lighten their hair.

In current work, the problem of the low durability of this coloring system has been addressed. In this context, it was found that the wash fastness of the coloring results obtained with pigments could be greatly improved by combining the pigments with certain amino-functionalized silicone polymers. In addition, the choice of particularly well-suited pigments and pigment concentrations on dark hair achieved a brighter color result so that lightening was even possible with this coloring system, which was possible until now only with oxidative hair treatment agents (bleaching or decolorizing agents).

Until now, there were still certain limits to the strength of this lightening. Thus, by using bright pigments in particularly high concentrations, it was indeed possible to achieve a lightening of one to two shades; it was, however, impossible to produce truly pure blond tones—in particular, on a very dark hair base such as dark brown or even almost black hair. In particular, low-damage lightening of very dark hair is, however, the strong desire of many users. However, the conventional pigment lightening agents known from the prior art have been previously unable to satisfy this wish.

### SUMMARY OF THE INVENTION

It was the object of the present invention to provide an agent based upon pigments with particularly strong lightening performance. By using the lightening agent, it should be possible to create a true and light blond shade even on very dark hair bases so that users can lighten their hair blond for a day, for example, but can also remove this coloring without any residue after the desired period.

Surprisingly, it has been found that this aforementioned object can be achieved in an outstanding manner when keratinous fibers—in particular, human hair—is treated with an agent which contains at least one amino-functionalized silicone polymer (a1) and at least one white pigment (a2).

### DETAILED DESCRIPTION OF THE INVENTION

A first subject of the present invention is an agent for lightening keratinous material—in particular, human hair-containing:
(a1) at least one amino-functionalized silicone polymer, and
(a2) at least one white pigment.

During the work carried out for this invention, it was surprisingly found that, while coloring keratinous fibers, particularly bright and natural blond tones could be achieved if a lightening agent were applied which contained a combination of at least one amino-functionalized silicone polymer (a1) and at least one white pigment (a2).

### Keratinous Fibers

Keratinous fibers are understood to be wool, fur, and feathers and in particular human hair. Keratinous fibers are very particularly preferably understood to mean human hair.

### Agent for Lightening

In the context of this invention, the term, “agent for lightening,” is used for a coloring of the keratinous material—in particular, hair-brought about by use of pigments. In this coloring process, the pigments are deposited as coloring compounds together with the amino-functionalized silicone polymer(s) in a particularly homogeneous, uniform, and smooth film on the surface of the keratinous material. The lightening agent can in particular be used on keratinous fibers with a dark starting color. Due to the use of the white pigment(s), the coloring of the colored keratinous fibers is brighter than the starting color, so that a lightening effect or blonding effect is visually perceived. This lightening effect can be achieved without the otherwise customary oxidizing agents such as hydrogen peroxide and/or persulfate compounds, etc. The lightening agent according to the invention is a ready-to-use lightening agent.

### Amino-Functionalized Silicone Polymers (a1)

As a first ingredient (a1) essential to the invention, the agent contains at least one amino-functionalized silicone polymer. The amino-functionalized silicone polymer can alternatively also be referred to as aminosilicone or amodimethicone.

Silicone polymers are generally macromolecules with a molecular weight of at least 500 g/mol, preferably at least 1,000 g/mol, more preferably at least 2,500 g/mol, and more preferably at least 5,000 g/mol which comprise repeating organic units.

The maximum molecular weight of the silicone polymer depends upon the degree of polymerization (number of polymerized monomers) and the batch size, and is also determined by the polymerization method. In the context of the present invention, it is preferable if the maximum molecular weight of the silicone polymers is not more than 107 g/mol, preferably not more than 106 g/mol, and particularly preferably not more than 105 g/mol.

The silicone polymers comprise many Si—O repeat units, the Si atoms being able to bear organic groups such as, for example, alkyl groups or substituted alkyl groups. Alternatively, a silicone polymer is therefore also referred to as polydimethylsiloxane.

Corresponding to the high molecular weight of the silicone polymers, these are based upon more than 10 Si—O repeat units, preferably more than 50 Si—O repeat units, and particularly preferably more than 100 Si—O repeat units-very particularly preferably more than 500 Si—O units.

An amino-functionalized silicone polymer is understood to mean a functionalized silicone which bears at least one structural unit with an amino group. The amino-functionalized silicone polymer preferably bears a plurality of structural units with at least one amino group in each instance. An amino group is understood to mean a primary amino group, a secondary amino group, and a tertiary amino group. All these amino groups can be protonated in an acidic environment and are then present in their cationic form.

In principle, it was possible to achieve positive effects of amino-functionalized silicone polymers (a1) when they bore at least one primary, at least one secondary, and/or at least one tertiary amino group. However, dyeings with the highest color intensities were observed when an amino-functionalized silicone polymer (a1) was used in an agent containing at least one secondary amino group.

In an very particularly preferred embodiment, an agent according to the invention is characterized in that the agent contains at least one amino-functionalized silicone polymer (a1) with at least one secondary amino group.

The secondary amino groups(s) can be at different positions in the amino-functionalized silicone polymer. A particularly good effect was found when an amino-functionalized silicone polymer (a1) was used that had at least one, and preferably multiple, structural units of the formula (Si-amino).

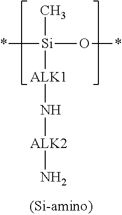

In the structural units of formula (Si-amino), the abbreviations ALK and ALK2 are each independently a linear or branched, divalent C₁-C₂₀ alkylene group.

In an additional very particularly preferred embodiment, an agent according to the invention is characterized in that the agent contains at least one amino-functionalized silicone polymer (a1) which comprises at least one structural unit of formula (Si-amino),

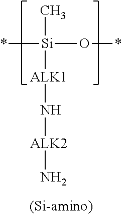

where ALK1 and ALK2 represent, independently of one another, a linear or branched, divalent C1-C20 alkylene group.

The position marked with an asterisk (*) always indicates the bond to other structural units of the silicone polymer. For example, the silicon atom adjacent to the asterisk can be bonded to an additional oxygen atom, and the oxygen atom adjacent to the asterisk can be bonded to an additional silicon atom or else to a C₁-C₆ alkyl group.

A divalent C₁-C₂₀ alkylene group can alternatively also be termed a double-bond C₁-C₂₀ alkylene group, which means that each moiety ALK1 or ALK2 can have two bonds.

In the case of ALK1, the silicon atom is bonded to the moiety ALK1, and the second bond is between ALK1 and the secondary amino group.

In the case of ALK2, the secondary amino group bonds with the moiety ALK2, and the second bond is formed between ALK2 and the primary amino group.

Examples of a linear divalent C₁-C₂₀ alkylene group are, for example, the methylene group (—CH₂—), the ethylene group (—CH₂—CH₂—), the propylene group (—CH₂—CH₂—CH₂—), and the butylene group (CH₂—CH₂—CH₂—CH₂—). The propylene group (—CH₂—CH₂—CH₂—) is particularly preferred. Starting at a chain length of 3 C atoms, divalent alkylene groups may also be branched. Examples of branched, divalent C₃-C₂₀ alkylene groups are (—CH₂—CH(CH₃)—) and (—CH₂—CH(CH₃)—CH₂—).

In an additional particularly preferred embodiment, the structural units of the formula (Si-amino) represent repeat units in the amino-functionalized silicone polymer (a1) so that the silicone polymer comprises multiple structural units of the formula (Si-amino).

In the following, particularly well-suited amino-functionalized silicone polymers (a1) with at least one secondary amino group are listed.

Color changes with particularly large differences in brightness were obtained when an agent was applied to the keratinous material which contains at least one amino-functionalized silicone polymer (a1) that comprises structural units of formula (Si-I) and formula (Si-II):

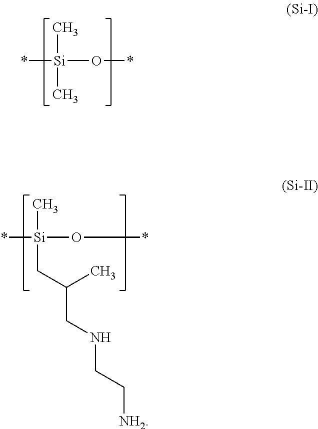

In an additional, explicitly very particularly preferred embodiment, an agent according to the invention is characterized in that it contains at least one amino-functionalized silicone polymer (a1) which comprises structural units of formula (Si-I) and formula (Si-II):

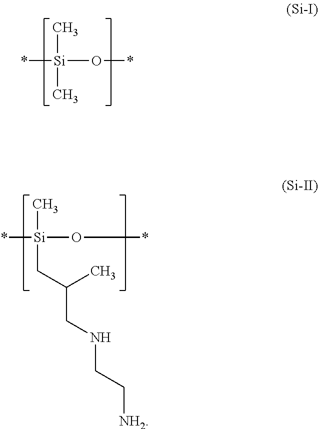

A corresponding amino-functionalized silicone polymer with the structural units (Si-I) and (Si-II) is, for example, the commercial product DC 2-8566 or Dowsil 2-8566 Amino Fluid which is sold commercially by Dow Chemical Company and which bears the designation, “Siloxanes and Silicones, 3-[(2-Aminoethyl)amino]-2-methylpropyl Me, Di-Me-Siloxane,” and the CAS number 106842-44-8. A further particularly preferred commercial product is Dowsil AP-8568 Amino Fluid, which is likewise sold commercially by Dow Chemical Company.

In another preferred embodiment, an agent according to the invention is characterized in that it contains at least one amino-functional silicone polymer (a1) of the formula of the formula (Si-III),

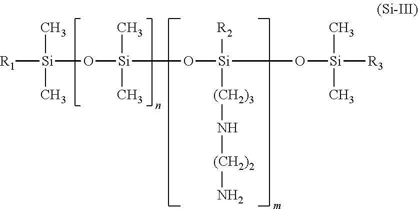

where
m and n denote numbers that are selected such that the sum (n+m) is in a range of 1 to 1,000,
n is a number in a range of 0 to 999, and m is a number in a range of 1 to 1,000,
R1, R2, and R3, which are identical or different, denote a hydroxyl group or a C1-4 alkoxy group,
at least one of the R1 to R3 groups denoting a hydroxyl group.

Additional agents preferred according to the invention are characterized by their content of at least one amino-functional silicone polymer (a1) of the formula (Si-IV),

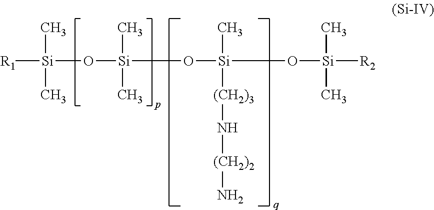

where
p and q denote numbers that are selected such that the sum (p+q) is in a range of 1 to 1,000,
p is a number in a range of 0 to 999, and q is a number in a range of 1 to 1,000, and
R1 and R2, which are different, denote a hydroxyl group or a C1-4 alkoxy group, at least one of the groups R1 to R2 denoting a hydroxyl group.

The silicones of the formulas (Si-III) and (Si-IV) differ by the grouping on the Si atom that carries the nitrogen-containing group: In formula (Si-III), R2 denotes a hydroxyl group or a C1-4 alkoxy group, whereas the group in formula (Si-IV) is a methyl group. The individual Si moieties, which are labeled with the indices m and n or p and q, need not be present as blocks; instead, the individual units can also be distributed randomly, i.e., in the formulas (Si-III) and (Si-IV), each R1-Si(CH₃)₂ group is not necessarily bound to a —[O—Si(CH₃)₂] moiety.

Agents according to the invention which contain at least one amino-functional silicone polymer (a1) of the formula of the formula (Si-V) have also proven to be particularly effective with respect to the desired effects:

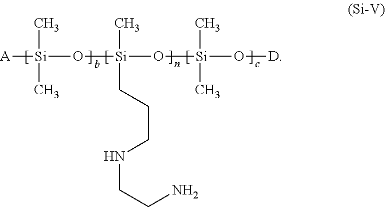

where
A represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group,
b, n, and c stand for integers between 0 and 1,000,
with the proviso that
   n>0 and b+c>0
   at least one of the conditions A=—OH or D=—H is met.

In the aforementioned formula (Si-V), the individual siloxane units having the indices b, c, and n are randomly distributed, i.e., they are not necessarily block copolymers.

The agent (a) can also comprise one or more different amino-functionalized silicone polymers which are described by the formula (Si-VI):

M(RₐQ_{b}SiO_{(4-a-b/2)X}(R_{c}SiO_{(4-c)/2)y}M (Si-VI)

In the above formula, R is a hydrocarbon or a hydrocarbon group having 1 to approximately 6 carbon atoms, Q is a polar group of general formula —R¹HZ, in which R¹ is a bivalent linking group bonded to hydrogen and the group Z, composed of carbon and hydrogen atoms, carbon, hydrogen, and oxygen atoms, or carbon, hydrogen, and nitrogen atoms, and Z is an organic, aminofunctional group containing at least one aminofunctional group; “a” assumes values in a range of approximately 0 to approximately 2, “b” assumes values in a range of approximately 1 to approximately 3, “a”+“b” is less than or equal to 3, and “c” is a number in a range of approximately 1 to approximately 3, and x is a number in a range of 1 to approximately 2,000, preferably approximately 3 to approximately 50, and most preferably approximately 3 to approximately 25, and y is a number in a range of approximately 20 to approximately 10,000, preferably approximately 125 to approximately 10,000, and most preferably approximately 150 to approximately 1,000, and M is a suitable silicone end group as is known in the prior art-preferably trimethylsiloxy. Non-limiting examples of the groups represented by R include alkyl groups, such as methyl, ethyl, propyl, isopropyl, isopropyl, butyl, isobutyl, amyl, isoamyl, hexyl, isohexyl, and the like; alkenyl groups such as vinyl, halovinyl, alkylvinyl, allyl, haloallyl, and alkylallyl; cycloalkyl groups such as cyclobutyl, cyclopentyl, cyclohexyl, and the like; phenyl groups; benzyl groups; halohydrocarbon groups such as 3-chloropropyl, 4-bromobutyl, 3,3,3-trifluoropropyl, chlorocyclohexyl, bromophenyl, chlorophenyl, and the like; and sulfur-containing groups such as mercaptoethyl, mercaptopropyl, mercaptohexyl, mercaptophenyl, and the like; R is preferably an alkyl group containing 1 to approximately 6 carbon atoms, and most preferably R is methyl. Examples of R¹ include methylene, ethylene, propylene, hexamethylene, decamethylene, —CH₂CH(CH₃) CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —(CH₂)₃C(O)SCH₂CH₂—.

Z is an organic, amino-functional group containing at least one functional amino group. A possible formula for Z is NH(CH₂)zNH₂, where z is 1 or more. Another possible formula for Z is —NH(CH₂)_{z}(CH₂)_{zz}NH, in which both z and zz, independently, are 1 or more, wherein this structure comprises diamino ring structures, such as piperazinyl. Z is most preferably a —NHCH₂CH₂NH₂ functional group. Another possible formula for Z is —N(CH₂)_{z}(CH₂)_{zz}NX₂ or —NX₂, where each X of X₂ is selected independently from the group consisting of hydrogen and alkyl groups having 1 to 12 carbon atoms, and zz is 0.

Q is most preferably a polar, amino-functional group of the formula —CH₂CH₂CH₂NHCH₂CH₂NH₂. In the formulas, “a” assumes values in the range of about 0 to about 2, “b” assumes values in the range of about 2 to about 3, “a”+“b” is less than or equal to 3, and “c” is a number in the range of about 1 to about 3. The molar ratio of the RₐQ_{b}SiO_{(4-a-b)/2} units to the R_{c}SiO_{(4-c)/2} units lies within a range of about 1:2 to 1:65, preferably about 1:5 to about 1:65, and most preferably about 1:15 to about 1:20. If one or more silicones of the above formula are used, then the various variable substituents in the above formula can be different in the various silicone components, present in the silicone mixture.

In the context of another preferred embodiment, an agent according to the invention is characterized in that it contains at least one amino-functional silicone polymer of the formula (Si-VII):

R′ₐG₃₋ₐ-Si(OSiG₂)ₙ-(OSiG_{b}R′_{2-b})ₘ—O—SiG₃₋ₐ-R′ₐ (Si-VII),

in which:
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃) CH₂CH₃, —CH(CH₃) CH₂CH₃, —O—C(CH₃)₃, or —C(CH₃)₃;
a represents a number between 0 and 3-in particular 0;
b represents a number between 0 and 1-in particular 1;
m and n are numbers whose sum (m+n) is between 1 and 2,000, and preferably between 50 and 150, n preferably assuming values of 0 to 1,999 and in particular from 49 to 149, and m preferably assuming values of 1 to 2,000, and in particular from 1 to 10;
R′ is a monovalent functional group selected from
-Q-N(R″)—CH₂—CH₂—N(R″)₂
-Q-N(R″)₂
-Q-N⁺(R″)₃A⁻
-Q-N⁺H(R″)₂A⁻
-Q-N⁺H₂(R″)A⁻
-Q-N(R″)—CH₂—CH₂—N⁺R″H₂A⁻,
   where each Q represents a chemical bond, —CH₂—, —CH₂—CH₂—, —CH₂CH₂CH₂—, —C(CH₃)₂—, —CH₂CH₂CH₂CH₂—, —CH₂C(CH₃)₂—, or —CH(CH₃) CH₂CH₂—,
   R″ representing identical or different functional groups from the group-H, —phenyl, —benzyl, —CH₂—CH(CH₃)Ph, from the C₁₋₂₀ alkyl functional groups-preferably-CH₃, —CH₂CH₃, —CH₂CH₂CH₃, —CH(CH₃)₂, —CH₂CH₂CH₂H₃, —CH₂CH(CH₃)₂, CH(CH₃) CH₂CH₃, —C(CH₃)₃, and A representing an anion preferably selected from chloride, bromide, iodide, or methosulfate.

In another preferred embodiment, an agent according to the invention is characterized in that it contains at least one amino-functional silicone polymer (a1) of the formula (Si-VIIa):

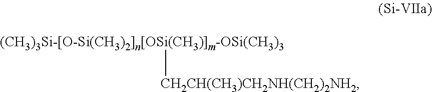

in which m and n are numbers of which the sum (m+n) is between 1 and 2,000, and preferably between 50 and 150, n preferably assuming values of 0 to 1,999 and in particular of 49 to 149 and m preferably assuming values of 1 to 2,000, and in particular of 1 to 10.

These silicones are designated as trimethylsilylamodimethicones in accordance with the INCI Declaration.

In another preferred embodiment, an agent according to the invention is characterized in that it contains at least one amino-functional silicone polymer of the formula (Si-VIIb):

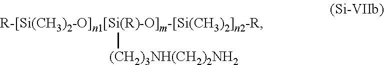

in which R represents —OH, —O—CH₃, or a —CH₃ group and m, n1, and n2 are numbers whose sum (m+n1+n2) is between 1 and 2,000, and preferably between 50 and 150, the sum (n1+n2) preferably assuming values of from 0 to 1,999 and in particular of 49 to 149, and m preferably assuming values of from 1 to 2,000, and in particular of 1 to 10.

According to the INCI Declaration, these amino-functionalized silicone polymers are referred to as amodimethicones.

Irrespective of which amino-functional silicones are used, agents according to the invention are preferred that contain an amino-functional silicone polymer (a1) of which the amine value is above 0.25 meq/g, preferably above 0.3 meq/g, and in particular above 0.4 meq/g. The amine value here represents the milliequivalents of amine per gram of the amino-functional silicone. Said value can be determined by titration and may also be given in the unit mg KOH/g.

Furthermore, agents which contained a specific 4-morpholinomethyl-substituted silicone polymer (a1) are also suitable. This amino-functionalized silicone polymer comprises structural units of formulas (Si-VIII) and of the formula (Si-IX):

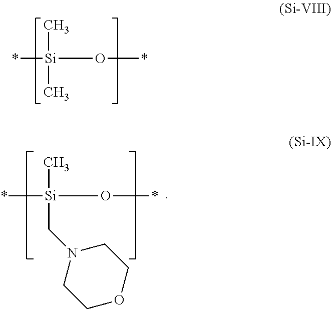

Corresponding 4-morpholinomethyl-substituted silicone polymers are described below.

A preferred amino-functionalized silicone polymer is known under the name f amodimethicone/morpholinomethyl silsesquioxane copolymer and is commercially available in the form of the raw material Belsil ADM 8301 E by Wacker.

For example, a silicone which has structural units of formulas (Si-VIII), (Si-IX), and (Si-X) can be used as 4-morpholinomethyl-substituted silicone:

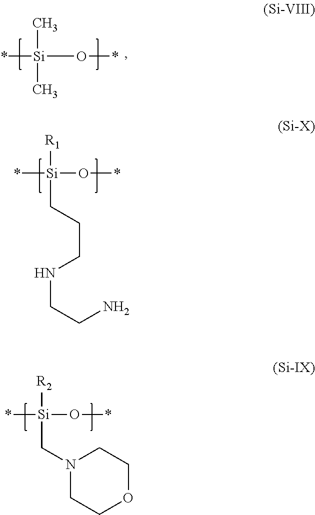

in which
R1 represents —CH₃, —OH, —OCH₃, —O—CH₂CH₃, —O—CH₂CH₂CH₃, or —O—CH(CH₃)₂;
R2 represents —CH₃, —OH, or —OCH₃.

Particularly preferred agents according to the invention contain at least one 4-morpholinomethyl-substituted silicone of formula (Si-XI)

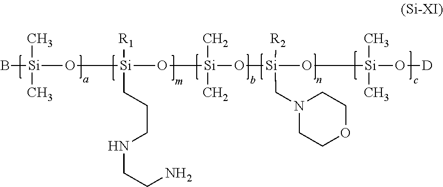

where
R1 represents —CH₃, —OH, —OCH₃, —O—CH₂CH₃, —O—CH₂CH₂CH₃, or —O—CH(CH₃)₂;
R2 represents —CH₃, —OH, or —OCH₃;
B represents an —OH, —O—Si(CH₃)₃, —O—Si(CH₃)₂OH, or —O—Si(CH₃)₂OCH₃ group;
D represents an —H, —Si(CH₃)₃, —Si(CH₃)₂OH, or —Si(CH₃)₂OCH₃ group;
a, b, and c represent, independently of one another, integers between 0 and 1,000, with the proviso that a+b+c>0;
m and n represent, independently of one another, integers between 1 and 1,000,
with the proviso that
   at least one of the conditions B=—OH or D=—H is met;
   the units a, b, c, m, and n are distributed randomly or in blocks in the molecule.

Structural formula (Si-XI) is intended to indicate that the siloxane groups n and m do not necessarily have to be directly bonded to an end group B or D. Instead, in preferred formulas (Si-VI), a>0 or b>0 and, in particularly preferred formulas (Si-VI), a>0 and c>0; i.e., the terminal group B or D is preferably bonded to a dimethylsiloxy group. In formula (Si-VI) as well, the siloxane units a, b, c, m, and n are preferably distributed randomly.

The silicones represented by formula (Si-VI) and used according to the invention can be trimethylsilyl-terminated (D or B=—Si(CH₃)₃), but they may also be dimethylsilylhydroxy-terminated dimethylsilylhydroxy- and dimethylsilylmethoxy-terminated at one end. In the context of the present invention, silicones which are particularly preferably used are selected from silicones in which:
B=—O—Si(CH₃)₂OH and D=—Si(CH₃)₃
B=—O—Si(CH₃)₂OH and D=—Si(CH₃)₂OH
B=—O—Si(CH₃)₂OH and D=—Si(CH₃)₂OCH₃
B=—O—Si(CH₃)₃ and D=—Si(CH₃)₂OH
B=—O—Si(CH₃)₂OCH₃ and D=—Si(CH₃)₂OH.

These silicones lead to enormous improvements in the hair properties of hair treated with the agents according to the invention, and to greatly improved protection during oxidative treatment.

It has been found to be particularly advantageous if the agent according to the invention contains the amino-functionalized silicone polymers (a1) in certain quantity ranges. Particularly good results were obtained when the agent contained-relative to the total weight of the agent-a total amount of 0.1 to 10.0 wt. %, preferably 0.2 to 8.0 wt. %, more preferably 0.3 to 6.0 wt. %, and very particularly preferably 0.4 to 5.0 wt. %.

In the context of another particularly preferred embodiment, an agent according to the invention is characterized in that it contains-relative to the total weight of the agent-one or more amino-functionalized silicone polymers (a1) in a total amount of 0.1 to 10.0 wt. %, preferably 0.2 to 8.0 wt. %, more preferably 0.3 to 6.0 wt. %, and very particularly preferably 0.4 to 5.0 wt. %.

### White Pigments (a2)

As a second ingredient essential to the invention, the agent according to the invention contains at least one white pigment. White pigments are uncolored inorganic pigments which preferably have a high refractive index.

White pigments are generally inorganic pigments whose optical effect is based predominantly upon light scattering independent of the wavelength. Organic white pigments hardly play a role in practice. Ideal white pigments do not exhibit any absorption in the range of visible light, but, rather, a high scattering capacity, which results in a high hiding power. The scattering capacity is all the greater the greater the difference between the refractive index of the white pigment and that of the surrounding medium.

In the context of another embodiment, particularly preferred is an agent for lightening keratinous fibers—in particular, human hair-containing:
(a1) at least one amino-functionalized silicone polymer, and
(a2) at least one inorganic white pigment.

From the Color Index, various white pigments are known to a person skilled in the art which are referred to as Pigment White, wherein this name is followed by a consecutive number. The Color Index (C.I. for short) has been a reference work for all conventional coloring agents and dye-based chemicals since 1925 and is considered a standard work in the field of pigment and dye chemistry.

| Color | | | | |
|---|---|---|---|---|
| Index | | | CAS | Refractive |
| Name | Pigment | Formula | number | index |
| Pigment | Lead white | Pb(OH)₂•2PBCO₃ | 1319-46-6 | 1.94-2.09 |
| White 1 | | | | |
| Pigment | Basic lead | PbSO₄ | 7446-14-2 | 1.93 |
| White 3 | sulfate | | | |
| Pigment | Zinc oxide | ZnO | 1314-13-2 | 2.02 |
| White 4 | | | | |
| Pigment | Lithopone | ZnS/BaSO₄ | 1345-05-7 | 1.84 |
| White 5 | | | | |
| Pigment | Titanium | TiO₂ | 13463-67-7 | 2.55 |
| White 6 | dioxide, | | | |
| | anatase | | | |
| Pigment | Titanium | TiO₂ | 13463-67-7 | 2.76 |
| White 6 | dioxide, | | | |
| | rutile | | | |
| Pigment | Zinc sulfide | ZnS | 1314-98-3 | 2.37 |
| White 7 | | | | |
| Pigment | Strontium | SrS | 1314-96-1 | 2.1 |
| White 8 | sulfide | | | |
| Pigment | Barium | BaCO₃ | 513-77-9 | 1.67 |
| White 10 | carbonate | | | |
| Pigment | Antimony | Sb₂O₃ | 1309-64-4 | 2.09-2.29 |
| White 11 | oxide | | | |
| Pigment | Zirconium | ZrO₂ | 1314-23-4 | 2.4 |
| White 12 | oxide | | | |
| Pigment | Bismuth | Bi₂(WO₄)₃ | 13595-87-4 | 2.17 |
| White 13 | tungstate | | | |
| Pigment | Bismuth | BiOCl | 7787-59-9 | 2.15 |
| White 14 | oxide | | | |
| | chloride | | | |
| Pigment | Stannous | SnO₂ | 18282-10-5 | 2 |
| White 15 | oxide | | | |
| Pigment | Bismuth | Bi₅O(OH)₉(NO₃)₄ | 1304-85-4 | |
| White 17 | subnitrate | | | |
| Pigment | Calcium | CaCO₃ | 471-34-1 | 1.58 |
| White 18 | carbonate | | | |
| | (chalk) | | | |
| Pigment | Calcium- | [Ca, Mg][CO₂]₂ | 16389-88-1 | 1.68 |
| White | magnesium | | | |
| 18:1 | carbonate | | | |
| | (dolomite) | | | |
| Pigment | Aluminum | Al₂Si₂H₄O₉ | 1332-58-7 | 1.55 |
| White 19 | silicate | | | |
| | (kaolin) | | | |
| Pigment | Mica | KAl₂(Si₃Al)O₁₀(OH, | 12001-26-2 | na = 1.560, |
| White 20 | (muscovite) | F)₂ | | nb = 1.594, |
| | | | | nc = 1.598 |
| Pigment | Barium | BaSO₄ | 7727-43-7 | 1.64 |
| White 21 | sulfate | | | |
| Pigment | Barium | BaSO₄ | 13462-86-7 | 1.64 |
| White 22 | (baryte) | | | |
| Pigment | Alumina | AlO₂ BaSO₄ | | 1.64 |
| White 23 | blanc fixe | | | |
| | (blancopone) | | | |
| Pigment | Aluminum | AlO₂ | 21645-51-2 | 1.57 |
| White 24 | hydroxide | | | |
| Pigment | Calcium | CaSO₄•2H₂O | 7778-18-9 | 1.59 |
| White 25 | sulfate | | | |
| | (gypsum) | | | |
| Pigment | Talc | Mg₂[Si₄O₁₀][OH]₂ | 14807-96-6 | 1.50-1.60 |
| White 26 | | | | |
| Pigment | Silica | SiO₂ | 7631-86-9 | 1.55 |
| White 27 | (Quartz) | | | |
| Pigment | Calcium | CaO₃Si | 10101-39-0 | 1.65 |
| White 28 | metasilicate | | | |
| | (wollastonite) | | | |
| Pigment | Zinc sulfate | ZnSO₄ | 7733-02-0 | 1.65 |
| White 32 | (white | | | |
| | copperas) | | | |

In the use in cosmetic lightening agents, particularly good lightening performance could be obtained with certain white pigments. Pigments selected from the group consisting of bismuth oxide chloride, boron nitride, synthetic fluorophlogopite, barium sulfate, calcium carbonate, calcium sulfate, zinc oxide, magnesium carbonate, titanium dioxide, zinc sulfide, lithopone, mica, and aluminum starch octenyl succinate have proven to be particularly suitable white pigments.

In the context of another particularly preferred embodiment, an agent according to the invention is characterized in that it contains at least one white pigment (a2) that is selected from the group of bismuth oxide chloride, boron nitride, synthetic fluorophlogopite, barium sulfate, calcium carbonate, calcium sulfate, zinc oxide, magnesium carbonate, titanium dioxide, zinc sulfide, lithopones, mica, and aluminum starch octenylsuccinate.

Bismuth oxide chloride is a white, crystalline powder and has the molecular formula BiOCl and bears the CAS number 7787-59-9. Alternative names are basic bismuth chloride, pigment white 14, and CI 77163. Bismuth oxide chloride occurs in anhydrous form and also as a hydrate such as BiOCl×H₂O.

Boron nitride has the molecular formula BN, bears the CAS number 10043-11-5, and is a white solid. Under the trade name of Boron Nitride HCJ 325, boron nitride can be commercially obtained, for example, from the company Sintec Keramik.

Synthetic fluorophlogopite is an artificial mineral with molecular formula (Mg₃K[AlF₂O(SiO₃)₃]) and with the CAS number 12003-38-2. Under the name of synthetic mica powder fluorphlogopite, the white powder can be purchased commercially—for example, from the company Jiangyin Youjia (Camida).

Barium sulfate is also referred to as barite or as Pigment White 21, has the CAS number 7727-43-7, and bears the molecular formula BaSO₄. Suppliers of barium sulfate are, for example, the companies Changzhou Jiaye Chemical, QGN Quimia Geral do Nordeeste, and Jiayehuagnog.

Calcium carbonate also bears the name of Pigment White 18 and has the molecular formula CaCO₃. The CAS number of calcium carbonate is 471-34-1. A source of calcium carbonate is the company Grolman and the company Chemische Fabrik Kalk.

Calcium sulfate is also known as Pigment White 25 and bears the CAS number 7778-18-9 (anhydrous). Gypsum is a white solid. The molecular formula of the anhydrous form is CaSO₄. The CAS number is 10034-76-1 for the hemihydrate and 10101-41-4 for the dihydrate. Both the anhydrous form and the hydrates of the gypsum are according to the invention.

Zinc oxide has the molecular formula ZnO and is also referred to as Pigment White 4. The CAS number of zinc oxide is 1314-13-2. Zinc oxide can, for example, be commercially obtained from the companies Indo Lysaght (Brenntag), EverZinc (formerly Umicore), or Avokal-Heller (Oqema).

Magnesium carbonate has the molecular formula MgCO3 and bears the CAS number 546-93-0. The CAS numbers for the hydrates are 13717-00-5 (monohydrate), 55-48-2 (dihydrate), 14457-83-1 (trihydrate), and 61042-72-6 (pentahydrate).

Titanium dioxide bears the molecular formula TiO2 and is also referred to as Pigment White 6. The CAS number of titanium dioxide is 13463-67-7. Titanium dioxide can be commercially obtained, for example, from the companies Univar, Anhui Gold Star Titanium Dioxide (Brenntag), or also from Merck.

Zinc sulfide also bears the name of Pigment White 7, has the molecular formula ZnS, and has the CAS number 1314-98-3. Zinc sulfide is a white powder and is commercially marketed, for example, by the companies Ventoroder Sachtleben Chemie.

Lithopone, which is also referred to as Pigment White 5, is a mixture of zinc sulfide and barium sulfate. Lithopone bears the CAS number 1345 May 7 and can be purchased commercially, for example, under the trade name of Lithopone Red Seal from the company Sachtleben Chemie.

A group of minerals from the branch of layered silicates is referred to as mica. An alternative name for said mica is Pigment White 20. Under the trade name of Mica MU M4, for example, a white powdered mica, which is muscovite and bears the CAS number 12001-26-2, can be purchased from the company Kaolins d′Arvor. Under the name of Mica 160, muscovite mica can be purchased as a white powder from the company Kings Mountain Mica.

Aluminum starch octenylsuccinate is a white powder with the CAS number 9087-61-0. Aluminum starch octenyl succinate can be purchased under the trade name, Dry Flo Pure or Dry Flo Plus—for example, from the companies Nouryon, AkzoNobel, or National Starch.

The white pigment(s) according to the invention can be used either as individual substances or in their mixture.

A particularly strong lightening effect and at the same time a particularly natural blond tone was achievable with the white pigment(s) selected from the group consisting of bismuth oxide chloride, boron nitride, synthetic fluorophlogopite, barium sulfate, calcium carbonate, calcium sulfate, zinc oxide, and magnesium carbonate. These white pigments are therefore explicitly very particularly preferred.

In the context of another explicitly very particularly preferred embodiment, an agent according to the invention is characterized in that it contains at least one white pigment (a2) selected from the group of bismuth oxide chloride, boron nitride, synthetic fluorophlogopite, barium sulfate, calcium carbonate, calcium sulfate, zinc oxide, and magnesium carbonate.

The white pigment(s) can be used as powders in the agent according to the invention. Furthermore, the use of the white pigments as a slurry or dispersion in a solvent and/or an oil is also according to the invention.

For example, bismuth oxychloride can be incorporated into the agent according to the invention in the form of its white powder. However, bismuth oxychloride can also be used in the form of a dispersion in ethylhexyl hydroxystearate. Both in powder form and in the form of the dispersion, the optical white impression of the pigment is retained.

The white pigment(s) (a2) are preferably used in specific quantity ranges in the agent according to the invention. Positive effects were obtained when the agent contained-relative to its total weight-one or more white pigments (a2) in a total amount of 0.1 to 10 wt. %, preferably 0.4 to 10.0 wt. %, more preferably 0.8 to 10.0 wt. %, and very particularly preferably 1.0 to 10.0 wt. %.

In the context of another particularly preferred embodiment, an agent according to the invention is characterized in that it contains—relative to its total weight—one or more white pigments (a2) in a total amount of 0.1 to 10 wt. %, preferably 0.4 to 10.0 wt. %, more preferably 0.8 to 10.0 wt. %, and very particularly preferably 1.0 to 10.0 wt. %.

### Color Pigments (a3)

For the development of shades or for fine-tuning the color appearance, the agent according to the invention can additionally contain one or more color pigments (a3) as a further optional component. The color pigments (a3) can be used, for example, if a reddish blond tone or a cool blond tone with a bluish color impression is to be created by applying the lightening agent.

Pigments in the sense of the present invention are understood to mean coloring compounds which have a solubility at 25° C. in water of less than 1.0 g/L, preferably less than 0.5 g/L, more preferably less than 0.1 g/L, and particularly preferably less than 0.05 g/L. The water solubility can be determined, for example, by means of the method described below: 0.5 g of the pigment is weighed into a beaker. A stir bar is added. Then one liter of distilled water is added. This mixture is heated to 25° C. while stirring with a magnetic stirrer for one hour. If still undissolved components of the pigment are visible in the mixture after this period, the solubility of the pigment is below 0.5 g/L. If the pigment-water mixture cannot be visually assessed due to the high intensity of the pigment that may be finely dispersed, the mixture is filtered. If a portion of undissolved pigments remains on the filter paper, the solubility of the pigment is below 0.5 g/L.

Suitable color pigments may be of inorganic and/or organic origin.

In a preferred embodiment, an agent according to the invention is characterized in that it contains at least one dying compound (a2) from the group consisting of inorganic and/or organic pigments.

Preferred color pigments are selected from synthetic or natural inorganic pigments. Inorganic color pigments of natural origin can be produced, for example, from chalk, ocher, umbra, green soil, burnt Sienna, or graphite. Furthermore, black pigments such as, for example, iron oxide black, chromatic pigments such as, for example, ultramarine or iron oxide red, and also fluorescent or phosphorescent pigments, can be used as inorganic color pigments.

Colored metal oxides, hydroxides and oxide hydrates, mixed phase pigments, sulfur-containing silicates, silicates, metal sulfides, complex metal cyanides, metal sulfates, chromates, and/or molybdates are particularly suitable. Particularly preferred color pigments are black iron oxide (CI 77499), yellow iron oxide (CI 77492), red and brown iron oxide (CI 77491), manganese violet (CI 77742), ultramarine (sodium aluminum sulphosilicates, Cl 77007, Pigment Blue 29), chromium oxide hydrate (CI77289), Iron Blue (ferric ferrocyanide, Cl77510), and/or carmine (cochineal).

Color pigments which are likewise particularly preferred according to the invention are colored pearlescent pigments. These are usually based on mica and may be coated with one or more metal oxides. Mica is a phyllosilicate. The most important representatives of these silicates are muscovite, phlogopite, paragonite, biotite, lepidolite, and margarite. In order to produce the pearlescent pigments in conjunction with metal oxides, mica—primarily muscovite or phlogopite,—is coated with a metal oxide.

As an alternative to natural mica, synthetic mica coated with one or more metal oxides(s) can also be used as a pearlescent pigment. Particularly preferred pearlescent pigments are based upon natural or synthetic mica and are coated with one or more of the aforementioned metal oxides. The color of the respective pigments can be varied by varying the layer thickness of the metal oxide(s).

In another preferred embodiment, an agent according to the invention is characterized in that it additionally contains a color pigment (a3) that is selected from the group of colored metal oxides, metal hydroxides, metal oxide hydrates, silicates, metal sulfides, complex metal cyanides, metal sulphates, bronze pigments, and/or from mica-based color pigments which are coated with at least one metal oxide and/or a metal oxychloride.

In another preferred embodiment, an agent according to the invention is characterized in that it contains at least one color pigment (a3) selected from mica-based pigments, which are coated with one or more metal oxides from the group consisting of titanium dioxide (CI 77891), black iron oxide (CI 77499), yellow iron oxide (CI 77492), red and/or brown iron oxide (CI 77491, CI 77499), manganese violet (CI 77742), ultramarine (sodium aluminum sulfosilicates, CI 77007, Pigment Blue 29), chromium oxide hydrate (CI 77289), chromium oxide (CI 77288), and/or iron blue (ferric ferrocyanide, CI 77510).

Examples of particularly suitable color pigments are commercially available, for example, under the trade names, Rona®, Colorona®, Xirona®, Dichrona®, and Timiron® from the company Merck, Ariabel® and Unipure® from the company Sensient, Prestige® from the company Eckart Cosmetic Colors, and Sunshine® from the company Sunstar.

Very particularly preferred color pigments with the trade name Colorona® are, for example:
Colorona Copper, Merck, MICA, CI 77491 (IRON OXIDES)
Colorona Passion Orange, Merck, Mica, CI 77491 (IRON OXIDES), Alumina
Colorona Patina Silver, Merck, MICA, CI 77499 (IRON OXIDES), CI 77891 (TITANIUM DIOXIDE)
Colorona RY, Merck, CI 77891 (TITANIUM DIOXIDE), MICA, CI 75470 (CARMINE)
Colorona Oriental Beige, Merck, MICA, CI 77891 (TITANIUM DIOXIDE), CI 77491 (IRON OXIDES) Colorona Dark Blue, Merck, MICA, TITANIUM DIOXIDE, FERRIC FERROCYANIDE
Colorona Chameleon, Merck, CI 77491 (IRON OXIDES), MICA
Colorona Aborigine Amber, Merck, MICA, CI 77499 (IRON OXIDES), CI 77891 (TITANIUM DIOXIDE)
Colorona Blackstar Blue, Merck, CI 77499 (IRON OXIDES), MICA
Colorona Patagonian Purple, Merck, MICA, CI 77491 (IRON OXIDES), CI 77891 (TITANIUM DIOXIDE), CI 77510 (FERRIC FERROCYANIDE)
Colorona Red Brown, Merck, MICA, CI 77491 (IRON OXIDES), CI 77891 (TITANIUM DIOXIDE)
Colorona Russet, Merck, CI 77491 (TITANIUM DIOXIDE), MICA, CI 77891 (IRON OXIDES)
Colorona Imperial Red, Merck, MICA, TITANIUM DIOXIDE (CI 77891), D&C RED NO. 30 (CI 73360)
Colorona Majestic Green, Merck, CI 77891 (TITANIUM DIOXIDE), MICA, CI 77288 (CHROMIUM OXIDE GREENS)
Colorona Light Blue, Merck, MICA, TITANIUM DIOXIDE (CI 77891), FERRIC FERROCYANIDE (CI 77510)
Colorona Red Gold, Merck, MICA, CI 77891 (TITANIUM DIOXIDE), CI 77491 (IRON OXIDES)
Colorona Gold Plus MP 25, Merck, MICA, TITANIUM DIOXIDE (CI 77891), IRON OXIDES (CI 77491)
Colorona Carmine Red, Merck, MICA, TITANIUM DIOXIDE, CARMINE
Colorona Blackstar Green, Merck, MICA, CI 77499 (IRON OXIDES)
Colorona Bordeaux, Merck, MICA, CI 77491 (IRON OXIDES)
Colorona Bronze, Merck, MICA, CI 77491 (IRON OXIDES)
Colorona Bronze Fine, Merck, MICA, CI 77491 (IRON OXIDES)
Colorona Fine Gold MP 20, Merck, MICA, CI 77891 (TITANIUM DIOXIDE), CI 77491 (IRON OXIDES)
Colorona Sienna Fine, Merck, CI 77491 (IRON OXIDES), MICA
Colorona Sienna, Merck, MICA, CI 77491 (IRON OXIDES)
Colorona Precious Gold, Merck, Mica, CI 77891 (Titanium dioxide), Silica, CI 77491 (IRON OXIDES), Tin oxide
Colorona Sun Gold Sparkle MP 29, Merck, MICA, TITANIUM DIOXIDE, IRON OXIDES, MICA, CI 77891, CI 77491 (EU)
Colorona Mica Black, Merck, CI 77499 (Iron oxides), Mica, CI 77891 (Titanium dioxide)
Colorona Bright Gold, Merck, Mica, CI 77891 (Titanium dioxide), CI 77491 (Iron oxides)
Colorona Blackstar Gold, Merck, MICA, CI 77499 (IRON OXIDES)

Additional particularly preferred color pigments with the trade name Xirona® are, for example:
Xirona Golden Sky, Merck, Silica, CI 77891 (Titanium Dioxide), Tin Oxide
Xirona Caribbean Blue, Merck, Mica, CI 77891 (Titanium Dioxide), Silica, Tin Oxide
Xirona Kiwi Rose, Merck, Silica, CI 77891 (Titanium Dioxide), Tin Oxide
Xirona Magic Mauve, Merck, Silica, CI 77891 (Titanium Dioxide), Tin Oxide.

In addition, particularly preferred color pigments with the trade name Unipure® are, for example:
Unipure Red LC 381 EM, Sensient CI 77491 (Iron Oxides), Silica
Unipure Black LC 989 EM, Sensient, CI 77499 (Iron Oxides), Silica
Unipure Yellow LC 182 EM, Sensient, CI 77492 (Iron Oxides), Silica.

In the context of another embodiment, the agent according to the invention can additionally contain one or more colored organic pigments (a3).

The organic pigments according to the invention are correspondingly insoluble, organic dyes or color lacquers, which may be selected, for example, from the group of nitroso, nitro, azo, xanthene, anthraquinone, isoindolinone, isoindoline, quinacridone, perinone, perylene, diketopyrrolopyrrole, indigo, thioindigo, dioxazine, and/or triarylmethane compounds.

Particularly well-suited organic pigments can for example include carmine, quinacridone, phthalocyanine, sorghum, blue pigments with the Color Index numbers CI 42090, CI 69800, CI 69825, CI 73000, CI 74100, or CI 74160, yellow pigments with the Color Index numbers CI 11680, CI 11710, CI 15985, CI 19140, CI 20040, CI 21100, CI 21108, CI 47000, or CI 47005, green pigments with the Color Index numbers CI 61565, CI 61570, or CI 74260, orange pigments with the Color Index numbers CI 11725, CI 15510, CI 45370, or CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, and/or CI 75470.

In another particularly preferred embodiment, an agent according to the invention is characterized in that it additionally contains at least one color pigment (a3) that is selected from the group consisting of carmine, quinacridone, phthalocyanine, sorghum, blue pigments with the Color Index numbers CI 42090, CI 69800, CI 69825, CI 73000, CI 74100, or CI 74160, yellow pigments with the Color Index numbers CI 11680, CI 11710, CI 15985, CI 19140, CI 20040, CI 21100, CI 21108, CI 47000, or CI 47005, green pigments with the Color Index numbers CI 61565, CI61570, or CI 74260, orange pigments with the Color Index numbers CI 11725, CI 15510, CI 45370, or CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, and/or CI 75470.

The organic pigment can also be a color lacquer. The term, color lacquer, in the sense of the invention is understood to mean particles which comprise a layer of absorbed dyes, with the unit consisting of particles and dye being insoluble under the above-mentioned conditions. The particles may, for example, be inorganic substrates which may be aluminum, silica, calcium borosilicate, calcium aluminum borosilicate, or aluminum.

For example, the alizarin color lacquer can be used as the color lacquer.

Owing to their excellent light and temperature resistance, the use of the aforementioned pigments in the method according to the invention is very particularly preferred. It is further preferred if the pigments used have a certain particle size. It is therefore advantageous according to the invention if the at least one pigment has a mean particle size D₅₀ of 1.0 to 50 μm, preferably 5.0 to 45 μm, preferably 10 to 40 μm, and in particular 14 to 30 μm. The mean particle size D50 can be determined, for example, using dynamic light scattering (DLS).

The color pigments (a3) are also preferably used in specific quantity ranges in the agent. Positive results were obtained when the agent contained-relative to the total weight of the agent-one or more color pigments (a3) in a total amount of 0.01 to 10.0 wt. %, preferably 0.1 to 5.0 wt. %, more preferably 0.2 to 2.5 wt. %, and very particularly preferably 0.25 to 1.5 wt. %.

In another very particularly preferred embodiment, an agent according to the invention is characterized in that the agent contains-relative to the total weight of the agent-one or more color pigments (a3) in a total amount of 0.01 to 10.0 wt. %, preferably 0.1 to 5.0 wt. %, more preferably 0.2 to 2.5 wt. %, and very particularly preferably 0.25 to 1.5 wt. %.

Weight Ratio (a2)/(a3)

In the agent according to the invention, the actual lightening effect is achieved by the white pigment(s) (a2), and the color pigments (a3) serve only for shading or fine adjustment of the desired blond shade. In order not to unduly reduce the lightening effect from excessive amounts of color pigments, the white pigment(s) (a2) are used in at least the same amount in comparison with the color pigments (a3). Preferably, the white pigments (a2) are used in a weight excess compared to the color pigments (a3).

Lightening of the keratinous fibers and at the same time the development of an extensive color palette of different blond shades was possible if the weight ratio of the total amount of white pigments (a2) contained in the agent to the total amount of color pigments (a3) contained in the agent, i.e., the weight ratio (a2)/(a3), was 10:1 to 1:1, preferably of 10:1 to 1.1:1, more preferably of 10:1 to 1.5:1, and particularly preferably of 10:1 to 2:1.

In the context of another very particularly preferred embodiment, an agent according to the invention is characterized in that it contains:
(a2) at least one white pigment, and
(a3) at least one color pigment,
   wherein the weight ratio of the total amount of white pigments (a2) contained in the agent to the total amount of color pigments (a3) contained in the composition, i.e., the weight ratio (a2)/(a3), is a value of 10:1 to 1:1, preferably of 10:1 to 1.1:1, more preferably of 10:1 to 1.5:1, and particularly preferably of 10:1 to 2:1.

In the case of a weight ratio of 10:1, the white pigments (a2) are used in a ten-fold weight excess compared to the color pigments (a3).

In the case of a weight ratio of 1.1:1, the white pigments (a2) are used in a 1.1-fold weight excess compared to the color pigments (a3).

White pigments (a2) and color pigments (a3) are very particularly preferably used in such amounts in the agent that the white pigments are present in the agent in a 2-fold to 10-fold weight excess compared to the color pigments.

The use of the following combinations of white pigment (a2) and color pigment (a3) has proven to be very particularly preferred:
bismuth oxide chloride/CI 71105, boron nitride/CI 71105, synthetic fluorophlogopite/CI 71105, barium sulfate/CI 71105, calcium carbonate/CI 71105, calcium sulfate/CI 71105, zinc oxide/CI 71105, magnesium carbonate/CI 71105,
bismuth oxide chloride/CI 15850, boron nitride/CI 15850, synthetic fluorophlogopite/CI 15850, barium sulfate/CI 15850, calcium carbonate/CI 15850, calcium sulfate/CI 15850, zinc oxide/CI 15850, magnesium carbonate/CI 15850,
bismuth oxide chloride/CI 69800, boron nitride/CI 69800, synthetic fluorophlogopite/CI 69800, barium sulfate/CI 69800, calcium carbonate/CI 69800, calcium sulfate/CI 69800, zinc oxide/CI 69800, magnesium carbonate/CI 69800,
bismuth oxide chloride/CI 19140, boron nitride/CI 19140, synthetic fluorophlogopite/CI 19140, barium sulfate/CI 19140, calcium carbonate/CI 19140, calcium sulfate/CI 19140, zinc oxide/CI 19140, magnesium carbonate/CI 19140,
bismuth oxide chloride/CI 19140, boron nitride/CI 19140, synthetic fluorophlogopite/CI 19140, barium sulfate/CI 19140, calcium carbonate/CI 19140, calcium sulfate/CI 19140, zinc oxide/CI 19140, and magnesium carbonate/CI 19140.

In the context of another very particularly preferred embodiment, an agent according to the invention is characterized in that it contains at least one combination of a white pigment (a2) and a color pigment (a3) which is selected from the group consisting of bismuth oxide chloride/CI 71105, boron nitride/CI 71105, synthetic fluorophlogopite/CI 71105, barium sulfate/CI 71105, calcium carbonate/CI 71105, calcium sulfate/CI 71105, zinc oxide/CI 71105, magnesium carbonate/CI 71105, bismuth oxide/CI 15850, boron nitride/CI 15850, synthetic fluorophlogopite/CI 15850, barium sulphate/CI 15850, calcium carbonate/CI 15850, calcium sulphate/CI 15850, zinc oxide/CI 15850, magnesium carbonate/CI 15850, bismuth oxychloride/CI 69800, boron nitride/CI 69800, synthetic fluorophlogopite/CI 69800, barium sulphate/CI 69800, calcium carbonate/CI 69800, calcium sulphate/CI 69800, zinc oxide/CI 69800, magnesium carbonate/CI 69800, bismuth oxide chloride/CI 19140, boron nitride/CI 19140, synthetic fluorophlogopite/CI 19140, barium sulphate/CI 19140, calcium carbonate/CI 19140, calcium sulphate/CI 19140, zinc oxide/CI 19140, magnesium carbonate/CI 19140, bismuth oxide chloride/CI 19140, boron nitride/CI 19140, synthetic fluorophlogopite/CI 19140, barium sulphate/CI 19140, calcium carbonate/CI 19140, calcium sulphate/CI 19140, zinc oxide/CI 19140, and magnesium carbonate/CI 19140.

In the case of an agent containing the combination of bismuth oxide chloride/CI 71105, both bismuth oxide chloride and the pigment with CI number 71105 are used in the lightening agent according to the invention.

### Water Content in the Agent

The agent described above is a ready-to-use agent which can be applied to the keratinous material. This ready-to-use agent preferably possesses a low to medium water content.

It has proved to be particularly expedient to limit the water content of the agent to a maximum of 50.0 wt. %, preferably 0.5 to 35.0 wt. %, more preferably 1.0 to 20.0 wt. %, and particularly preferably 1.5 to 15.0 wt. %. Therefore, the agents are particularly well suited which contain-relative to the total weight of the agent-0.1 to 50.0 wt. %, preferably 0.5 to 35.0 wt. %, more preferably 1.0 to 20.0 wt. %, and particularly preferably 1.5 to 15.0 wt. % water.

In another explicitly very particularly preferred embodiment, an agent according to the invention is characterized in that it contains-relative to the total weight of the agent-0.1 to 50.0 wt. %, preferably 0.5 to 35.0 wt. %, more preferably 0.1 to 20.0 wt. %, and especially preferably 1.5 to 15.0 wt. % water.

A maximum water content of 15 wt. % in the agent means, for example, that the amount of water in the agent, which is caused by the direct use of water in the agent and/or which is introduced into the agent by the amounts of water present in the utilized ingredients, is no more than 15 wt. %. The indication of the amount of water in percent by weight is relative to the total weight of the agent.

### Solvent in the Agent

The use of solvents in the agent has led to very good results. For this reason, the agent according to the invention can additionally contain at least one solvent as an optional component.

Suitable solvents that can be used are, for example, solvents from the group consisting of 1,2-propylene glycol, 1,3-propylene glycol, 1,2-butylene glycol, dipropylene glycol, ethanol, isopropanol, diethylene glycol monoethyl ether, glycerol, phenoxyethanol, and benzyl alcohol. The use of 1,2-propylene glycol is very particularly preferred.

In another very particularly preferred embodiment, an agent according to the invention is characterized in that it contains at least one solvent from the group consisting of 1,2-propylene glycol, 1,3-propylene glycol, ethylene glycol, 1,2-butylene glycol, dipropylene glycol, ethanol, isopropanol, diethylene glycol monoethyl ether, glycerol, phenoxyethanol, and benzyl alcohol, and very particularly preferably 1,2-propylene glycol.

Alternatively, 1,2-propylene glycol is also referred to as 1,2-propanediol and bears the CAS numbers 57-55-6 [(RS)-1,2-dihydroxypropane], 4254-14-2 [(R)-1,2-dihydroxypropane], and 4254-153 [(S)-1,2-dihydroxypropane]. Ethylene glycol is alternatively also referred to as 1,2-ethanediol and bears the CAS number 107-21-1. Glycerol is alternatively also referred to as 1,2,3-propanetriol and bears the CAS number 56-81-5. Phenoxyethanol has the CAS number 122-99-6.

All the solvents described above are commercially available from various chemicals suppliers such as Aldrich or Fluka.

By using the abovementioned solvents in suitable use quantities, a particularly stable agent can be obtained with which color results on the keratinous material of an especially high intensity can be obtained.

The solvent(s) are preferably contained in the agent according to the invention in certain quantity ranges. In this context, it has been found to be preferable if the agent contains one or more solvents different from water in a total amount of 5.0 to 60.0 wt. %, preferably 10.0 to 50.0 wt. %, and particularly preferably 20 to 50 wt. %, relative to the total weight of agent.

In another particularly preferred embodiment, an agent according to the invention is characterized in that it contains-relative to the total weight of the agent
one or more solvents from the group consisting of 1,2-propylene glycol, 1,3-propylene glycol, ethylene glycol, 1,2-butylene glycol, dipropylene glycol, ethanol, isopropanol, diethylene glycol monoethyl ether, glycerol, phenoxyethanol, and benzyl alcohol in a total amount of 5.0 to 60.0 wt. %, preferably 10.0 to 50.0 wt. %, and very particularly preferably 20 to 50 wt. %.

### Alkylene Glycols in the Agent

In addition to the solvents described above, or instead of them, the agent according to the invention can also contain one or more alkylene glycols. For example, very good results were obtained if the agent contained at least one alkylene glycol of the formula (AG-1),

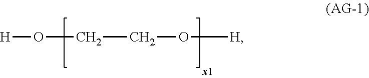

where x1 is an integer from 1 to 100.

The alkylene glycols of formula (AG-1) are protic substances having at least two hydroxyl groups which, due to their repeating —CH₂—CH₂—O— unit, can also be referred to as polyethylene glycols insofar as x1 is a value of at least 2. In the alkylene glycols of formula (AG-1), x1 is an integer from 1 to 100. In the context of the work that led to this invention, it was found that these polyethylene glycols, together with the other constituents of the agent, are particularly well suited for ensuring sufficiently intense dyeing and for improving the feel of the keratinous fibers.

Depending upon their chain length, polyethylene glycols are liquid or solid water-soluble polymers. Polyethylene glycols with a molecular mass between 200 g/mol and 400 g/mol are non-volatile liquids at room temperature. PEG 600 has a melting range of 17 to 22° C. and therefore a pasty consistency. With molecular masses above 3,000 g/mol, the PEG's are solid substances and are commercially available as flakes or powders.

The use of low molecular weight alkylene glycols (or polyethylene glycols) has proven to be well suited for achieving the object according to the invention. In the event of low molecular weight alkylene glycols (or, respectively, polyethylene glycols) in the context of the present invention, x1 denotes an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15.

In another very particularly preferred embodiment, an agent according to the invention is characterized in that it contains at least one alkylene glycol of formula (AG-1),

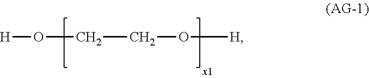

where x1 denotes an integer from 1 to 100, preferably an integer from 2 to 60, more preferably an integer from 3 to 40, even more preferably an integer from 4 to 20, and very particularly preferably an integer from 6 to 15.

A very particularly preferred low molecular weight polyethylene glycol is PEG-8, for example. PEG-8 comprises, on average, 8 ethylene glycol units (x1=8), has an average molecular weight of 400 g/mol, and bears the CAS number 25322-68-3. PEG-8 is alternatively also referred to as PEG 400 and is commercially available, for example, from APS.

Additional well-suited low molecular weight polyethylene glycols are, for example, PEG-6, PEG-7, PEG-9, and PEG-10.

Another well-suited polyethylene glycol is PEG-32, for example. PEG-32 comprises 32 ethylene glycol units (x1=32), has a mean molar mass of 1,500 g/mol, and bears the CAS number 25322-68-3. PEG-32 is alternatively also referred to as PEG 1500 and can, for example, be purchased commercially from Clariant.

Furthermore, very good results were obtained if the agent contained at least one alkylene glycol of the formula (AG-2),

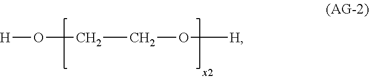

where x2 is an integer from 1,001 to 95,000.

The alkylene glycols of the formula (AG-2) are polyethylene glycols with a very high molecular weight, in which the index number x2 represents an integer from 1,001 to 95,000.

In combination, the alkylene glycols (AG-1) and (AG-2) allow a good applicability of the cosmetic agent, a good dye absorption of white pigment (a2) and aminosilicone (a1), and in particular also an improvement in the tactile sensation of the keratinous fibers.

In the case of particularly preferred alkylene glycols of the formula (AG-2), x2 represents an integer from 1,001 to 80,000, preferably from 1,400 to 40,000, more preferably from 1,800 to 20,000, and very particularly preferably from 2,000 to 10,000.

In another very particularly preferred embodiment, an agent according to the invention is characterized in that it contains at least one alkylene glycol of formula (AG-2),

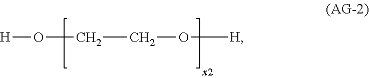

where x2 represents an integer from 1,001 to 80,000, preferably from 1,400 to 40,000, more preferably from 1,800 to 20,000, and very particularly preferably from 2,000 to 10,000.

Very particularly well-suited high-molecular-weight polyethylene glycols are the commercial products commercially available under the trade name, Polyox from Dow.

For example, POLYOX WSR 301 is well suited; in this polyethylene glycol of formula (AG-2), x2 is on average an integer of 90,000. POLYOX WSR 301 has a weight-average molecular weight of 4,000,000 daltons.

POLYOX WSR N-60K, which is alternatively also referred to as PEG-45M, is also well suited. In this polyethylene glycol of formula (AG-2), X2 has an average value of 45,000. PEG-45M has a weight-average molecular weight of 2,000,000 daltons.

POLYOX WSR N-12K, which is alternatively also referred to as PEG-23M, is also well suited. In this polyethylene glycol of formula (AG-2), x2 is on average an integer of 23,000. PEG-23M has a weight-average molecular weight of 1,000,000 daltons.

POLYOX WSR-1105 is also well suited; in this polyethylene glycol of the formula (AG-2), x2 stands for an average integer of 20,454, and the average molecular weight is 900,000 daltons.

POLYOX WSR 35 is also well suited; in this polyethylene glycol of formula (AG-2), x2 is on average an integer of 4,500. POLYOX WSR 35 has a weight-average molecular weight of 200,000 daltons.

POLYOX WSR-10 is very particularly well suited; in this polyethylene glycol of formula (AG2), x2 is on average an integer of 2,272. POLYOX WSR N-10 has a weight-average molecular weight of 100,000 daltons.

Furthermore, very good results were obtained if the agent contained at least one alkylene glycol of the formula (AG-3), wherein the alkylene glycol of the formula (AG-3) is a polyethylene glycol having an average molecular weight.

In the optionally contained alkylene glycol of formula (AG-3),

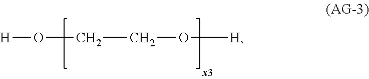

x3 denotes an integer from 101 to 1,000, preferably an integer from 105 to 800, more preferably an integer from 107 to 600, even more preferably an integer from 109 to 400, and very particularly preferably an integer from 110 to 200.

In another very particularly preferred embodiment, an agent according to the invention is characterized in that it contains at least one alkylene glycol of formula (AG-3),

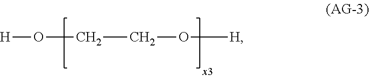

where x3 denotes an integer from 101 to 1,000, preferably an integer from 105 to 800, more preferably an integer from 107 to 600, even more preferably an integer from 109 to 400, and very particularly preferably an integer from 110 to 200.

A very particularly well-suited polyethylene glycol with an average molecular weight is, for example, PEG 6000, which can be obtained commercially from the National Starch company (China). The molecular weight of PEG 6000 is 6,000 to 7,500 g/mol, corresponding to an x3 value of 136 to 171.

Another well-suited polyethylene glycol is PEG 12000 which, for example, is commercially sold by CG chemicals under the trade name of Polyethylene Glycol 12000 S (or PEG 12000 S). The molecular weight of PEG 12000 is given as 10,500 to 15,000 g/mol, corresponding to an x3 value of 238 to 341.

Another well-suited polyethylene glycol is also PEG 20000 which is commercially available under the trade name, Polyglycol 20000 P or under the alternative name, PEG-350 from Clariant. For PEG 20000, an average molecular weight of 20,000 g/mol is given, which corresponds to an x3 value of 454.

The alkylene glycols of the formula (AG-1), (AG-2), and/or (AG-3) are also preferably contained in certain quantity ranges in the agent according to the invention. It has been found to be particularly advantageous if the agent contains, relative to its total weight, one or more alkylene glycols-preferably one or more alkylene glycols of the formulas (AG-1), (AG-2), and/or (AG-3)—in a total amount of 1.0 to 90.0 wt. %, preferably 10.0 to 80.0 wt. %, more preferably 15.0 to 70.0 wt. %, and very particularly preferably 30.0 to 65.0 wt. %.

In another very particularly preferred embodiment, an agent according to the invention is characterized in that it contains, relative to the total weight of the agent, one or more alkylene glycols-preferably one or more alkylene glycols of the formulas (AG-1), (AG-2), and/or (AG-3)—in a total amount of 1.0 to 90.0 wt. %, preferably 10.0 to 80.0 wt. %, more preferably 15.0 to 70.0 wt. %, and most preferably 30.0 to 65.0 wt. %.

### Additional Ingredients in the Agent

The agent according to the invention may also contain even more active ingredients, auxiliaries, and additives, such as structurants like fatty components, glucose, maleic acid, and lactic acid; hair-conditioning compounds like phospholipids—for example, lecithin and kephalins; perfume oils; dimethyl isosorbide and cyclodextrins; polymers such as anionic, non-ionic, and cationic polymers; surfactants such as anionic, non-ionic, cationic, zwitterionic, and amphoteric surfactants; fatty components; fiber structure-improving active ingredients—in particular, mono-, di-, and oligosaccharides such as glucose, galactose, fructose, fruit sugar, and lactose; dyes for lightening the agent; anti-dandruff active ingredients such as piroctone olamine, zinc omadine, and climbazole; amino acids and oligopeptides; protein hydrolysates based upon animals and/or plants, as well as in the form of their fatty acid condensation products or optionally anionically or cationically modified derivatives; vegetable oils; light stabilizers and UV blockers; active ingredients such as panthenol, pantothenic acid, pantolactone, allantoin, pyrrolidinone carboxylic acids and their salts, and bisabolol; polyphenols—in particular, hydroxycinnamic acids, 6,7-dihydroxycoumarins, hydroxybenzoic acids, catechins, tannins, leucoanthocyanidins, anthocyanidins, flavanones, flavones, and flavonols; ceramides or pseudoceramides; vitamins, provitamins, and vitamin precursors; plant extracts; fats and waxes such as fatty alcohols, beeswax, montan wax, and paraffins; swelling and penetrating agents such as glycerol, propylene glycol monoethyl ether, carbonates, hydrogen carbonates, guanidines, ureas, and primary, secondary, and tertiary phosphates; opacifiers such as latex, styrene/PVP, and styrene/acrylamide copolymers; pearlescent agents such as ethylene glycol mono- and distearate and PEG-3 distearate; and propellants such as propane-butane mixtures, N₂O, dimethyl ether, CO₂, and air.

The selection of these additional substances is made by the person skilled in the art according to the desired properties of the agents. With respect to other optional components and the employed amounts of said components, reference is made expressly to relevant manuals known to the person skilled in the art. The additional active ingredients and auxiliaries are used in the preparations according to the invention preferably always in amounts of 0.0001 to 25 wt. %, and in particular of 0.0005 to 15 wt. %, relative to the total weight of the particular agent.

### Method for Lightening Keratinous Fibers

The agents described above can be used outstandingly in methods for lightening keratinous fibers—in particular, human hair.

A second subject of the present invention is therefore a method for lightening keratinous fibers—in particular, human hair-comprising the following steps:
(1) applying an agent as disclosed in detail in the description of the first subject of the invention to the keratinous fibers,
(2) letting the agent act upon the keratinous fibers, and
(3) optionally rinsing the agent with water.

In step (1) of the method according to the invention, the lightening agent of the first subject of the invention is applied to the keratinous fibers, which are most preferably human hair.

In step (2) of the method according to the invention, the agent is then allowed to act upon the keratinous fibers after its application. In this context, various exposure times of, for example, 30 seconds to 60 minutes are conceivable.

However, a great advantage of the color changing system according to the invention is that an intensive color result can be achieved even in very short periods after short exposure times. For this reason, it is advantageous for the application mixture to remain on the keratinous fibers after application only for comparatively short periods of 30 seconds to 15 minutes, preferably 30 seconds to 10 minutes, and particularly preferably 1 to 5 minutes.

In a further preferred embodiment, a method according to the invention is characterized by
(2) exposing the keratinous fibers to the agent for a period of 30 seconds to 15 minutes, preferably 30 seconds to 10 minutes, and more preferably 1 to 5 minutes.

After the action of the lightening agent on the keratinous fibers, said keratinous fibers are rinsed with water in step (3) of the method.

In one embodiment, the lightening agent can be washed out with water only, i.e., without the aid of an after-treatment agent or a shampoo. The application of an after-treatment agent or conditioner in a subsequent step (3) or after step (3) is also conceivable in principle.

Concerning the additional preferred embodiments of the method according to the invention, what has been said about the agent according to the invention applies mutatis mutandis.

### Examples

The following formulation was produced (all information is in wt. % unless indicated otherwise).

| ready-to-use lightening agent | (wt. %) |
|---|---|
| DOWSIL AP-8568 Amino | 0.75 |
| Fluid (aminosilicone (a1)) | |
| Hexamethyldisiloxane | 6.0 |
| 1,2-propanediol | 30.0 |
| Water | 10.0 |
| Plantacare 2000 UP (decyl | 0.2 |
| glucoside, 51-55% in water) | |
| Boron nitride (a2) | 1.0 |
| Polyethylene glycol (PEG 6000, | 10.0 |
| molar weight 6,000 g/mol) | |
| Polyethylene glycol (PEG 400, | up to 100 |
| molar weight 400 g/mol) | |

### 2. Coloration

Hair strands (Kerling 4-0, dark blond) were measured colorimetrically. The ready-to-use lightening agent was then applied to the hair strands (liquor ratio: 1 g of agent per 1 g of hair strand) and allowed to act for three minutes. Subsequently, the strands of hair were washed thoroughly (1 minute) with water and then dried with a hand towel. Subsequently, the strands were again measured colorimetrically.

The dE value used for assessing color difference is derived from the L*a*b* colorimetric values measured at the respective strands as follows:

$$dE = {[ {( {L}_{i} - {L}_{0} )}^{2} + {( {a}_{i} - {a}_{0} )}^{2} + ( {b}_{i} - {b}_{0} ) ]}^{1} {/}^{2}$$

L₀, a₀, and b₀=measured values of untreated strands
Lᵢ, aᵢ, and bᵢ=measured values of the lightened strands.

The greater the dE value, the greater the color distance between the untreated hair and the lightened hair, and the stronger the color change.

The dL value used to assess the lightness results from the L value measured at the respective strands as follows:

$$dL = {L}_{i} - {L}_{0}$$

L₀=measured value of the untreated strands
Lᵢ=measured value of the lightened strands.

The greater the dL value, the greater the difference in brightness compared to the untreated hair and the stronger the lightening effect.

The following color results were obtained:

| | | | | dE | dL |
|---|---|---|---|---|---|
| | | | | (comparison | (comparison |
| | | | | before | before |
| | | | | and after | and after |
| | L | a | b | lightening) | lightening) |
| Kerling 4-0 prior | 20.05 | 3.99 | 5.37 | — | — |
| to application of | | | | | |
| the lightening | | | | | |
| agent | | | | | |
| Kerling 4-0 after | 29.89 | 2.23 | 1.13 | 10.86 | 9.85 |
| application of | | | | | |
| the lightening | | | | | |
| agent | | | | | |

What is claimed is:

1. An agent for lightening keratinous fibers, comprising:
(a1) at least one amino-functionalized silicone polymer, and
(a2) at least one white pigment.

2. The agent according to claim 1, wherein the at least one amino-functionalized silicone polymer (a1) comprises at least one structural unit of the formula (Si-amino), 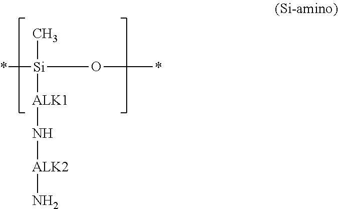
where ALK1 and ALK2 represent, independently of one another, a linear or branched, divalent C₁-C₂₀ alkylene group.

3. The agent according to claim 1, wherein the at least one amino-functionalized silicone polymer (a1) comprises structural units of the formula (Si-I) and of the formula (Si-II),

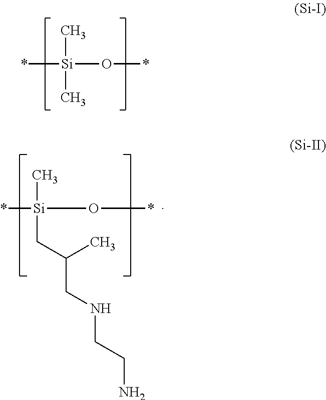

4. The agent according to claim 1, wherein, relative to the total weight of the composition, the one or more amino-functionalized silicone polymers (a1) is present in the agent in a total amount of 0.1 to 10.0 wt. %.

5. The agent according to claim 1, wherein, relative to the total weight of the composition, the one or more amino-functionalized silicone polymers (a1) is present in the agent in a total amount of 0.3 to 6.0 wt. %.

6. The agent according to claim 1, wherein, relative to the total weight of the composition, the one or more amino-functionalized silicone polymers (a1) is present in the agent in a total amount of 0.4 to 5.0 wt. %.

7. The agent according to claim 1, wherein at least one white pigment (a2) is selected from the group consisting of: bismuth oxide chloride, boron nitride, synthetic fluorophlogopite, barium sulfate, calcium carbonate, calcium sulfate, zinc oxide, magnesium carbonate, titanium dioxide, zinc sulfide, lithopones, mica, and aluminum starch octenylsuccinate.

8. The agent according to claim 1, wherein, relative to the total weight thereof, the one or more white pigments (a2) is present in the agent in a total amount of 0.1 to 10 wt. %.

9. The agent according to claim 1, wherein, relative to the total weight thereof, the one or more white pigments (a2) is present in the agent in a total amount of 0.8 to 10 wt. %.

10. The agent according to claim 1, wherein, relative to the total weight thereof, the one or more white pigments (a2) is present in the agent in a total amount of 1.0 to 10 wt. %.

11. The agent according claim 1, further comprising at least a color pigment (a3), said color pigment selected from the group consisting of: colored metal oxides, metal hydroxides, metal oxide hydrates, silicates, metal sulfides, complex metal cyanides, metal sulfates, bronze pigments, and from mica-based color pigments which are coated with at least one metal oxide and/or a metal oxychloride.

12. The agent according to claim 1, further comprising at least a color pigment (a3) selected from the group consisting of: carmine, quinacridone, phthalocyanine, sorghum, blue pigments with the Color Index numbers Cl 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915, CI 75470.

13. The agent according to claim 1, further comprising at least one color pigment (a3), wherein the weight ratio of the total amount of white pigments (a2) in the agent to the total amount of color pigments (a3) in the agent (a2)/(a3) is 10:1 to 1:1.

14. The agent according to claim 1, further comprising (a3) at least one color pigment (a3), wherein the weight ratio of the total amount of white pigments (a2) in the agent to the total amount of color pigments (a3) in the agent (a2)/(a3) is 10:1 to 1.5:1.

15. The agent according to claim 1, further comprising at least one color pigment (a3), wherein the weight ratio of the total amount of white pigments (a2) in the agent to the total amount of color pigments (a3) in the agent (a2)/(a3) is 10:1 to 2:1.

16. The agent according to claim 1, further comprising at least one color pigment (a3), wherein the agent includes at least one combination of a white pigment (a2) and a color pigment (a3) which is selected from the group consisting of bismuth oxide chloride/CI 71105, boron nitride/CI 71105, synthetic fluorophlogopite/CI 71105, barium sulfate/CI 71105, calcium carbonate/CI 71105, calcium sulfate/CI 71105, zinc oxide/CI 71105, magnesium carbonate/CI 71105, bismuth oxide/CI 15850, boron nitride/CI 15850, synthetic fluorophlogopite/CI 15850, barium sulphate/CI 15850, calcium carbonate/CI 15850, calcium sulphate/CI 15850, zinc oxide/CI 15850, magnesium carbonate/CI 15850, bismuth oxychloride/CI 69800, boron nitride/CI 69800, synthetic fluorophlogopite/CI 69800, barium sulphate/CI 69800, calcium carbonate/CI 69800, calcium sulphate/CI 69800, zinc oxide/CI 69800, magnesium carbonate/CI 69800, bismuth oxide chloride/CI 19140, boron nitride/CI 19140, synthetic fluorophlogopite/CI 19140, barium sulphate/CI 19140, calcium carbonate/CI 19140, calcium sulphate/CI 19140, zinc oxide/CI 19140, magnesium carbonate/CI 19140, bismuth oxide chloride/CI 19140, boron nitride/CI 19140, synthetic fluorophlogopite/CI 19140, barium sulphate/CI 19140, calcium carbonate/CI 19140, calcium sulphate/CI 19140, zinc oxide/CI 19140, and magnesium carbonate/CI 19140.

17. The agent according claim 1, further comprising one or more solvents, said one or more solvents being selected from the group consisting of: 1,2-propylene glycol, 1,3-propylene glycol, ethylene glycol, 1,2-butylene glycol, dipropylene glycol, ethanol, isopropanol, diethylene glycol monoethyl ether, glycerol, phenoxyethanol, and benzyl alcohol.

18. The agent according to claim 1, further comprising at least one alkylene glycol of the formula (AG-1), 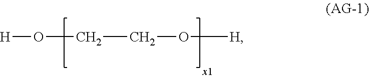
where x1 denotes an integer from 1 to 100.

19. The agent according claim 1, further comprising at least one alkylene glycol of the formula (AG-3), 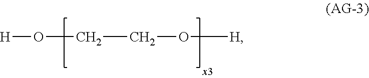
where x3 denotes an integer from 101 to 1,000.

20. A method for lightening keratinous fibers, comprising,
(1) applying an agent according to claim 1 to the keratinous fibers,
(2) letting the agent act upon the keratinous fibers, and
(3) optionally rinsing the agent with water.
